SEQUENCE LISTING (8 sequences)

SEQ ID NO: 1 - protein, 5 aa, Artificial Sequence
  PEPTIDE (1)..(5): X is any amino acid
  PEPTIDE (1)..(5): Synthesized Peptide
GPRPX

SEQ ID NO: 2 - protein, 5 aa, Artificial Sequence
  PEPTIDE (1)..(5)
  PEPTIDE (1)..(5): Synthesized Peptide
GPRPK

SEQ ID NO: 3 - protein, 4 aa, Artificial Sequence
  PEPTIDE (1)..(4): X is any amino acid
  PEPTIDE (1)..(4): Synthesized Peptide
GPRX

SEQ ID NO: 4 - protein, 4 aa, Artificial Sequence
  PEPTIDE (1)..(4)
  PEPTIDE (1)..(4): Synthesized Peptide
GPRS

SEQ ID NO: 5 - protein, 4 aa, Artificial Sequence
  PEPTIDE (1)..(4)
  PEPTIDE (1)..(4): Synthesized Peptide
GPRV

SEQ ID NO: 6 - protein, 3 aa, Artificial Sequence
  PEPTIDE (1)..(3)
  PEPTIDE (1)..(3): Synthesized Peptide
RGD

SEQ ID NO: 7 - protein, 5 aa, Artificial Sequence
  PEPTIDE (1)..(5)
  PEPTIDE (1)..(5): Synthesized Peptide
EHIPA

SEQ ID NO: 8 - protein, 9 aa, Artificial Sequence
  PEPTIDE (1)..(9)
  PEPTIDE (1)..(9): Synthesized Peptide
GPRPERHES

(19) United States
(12) Patent Application Publication
(10) Pub. No.: US 2007/0253967 A1
(43) Pub. Date: Nov. 1, 2007

(54) Agent for the treatment and/or prophylaxis of microcirculatory disorders

(76) Inventors: W.O. Richter, Windach-Schoffelding (DE); Robert Koll, Kirchheim (DE); Franz Bieber, Guysborough (CA); W. Tschope, Unterschleissheim (DE)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(21) Appl. No.: 11/784,196

(22) Filed: Apr. 5, 2007

Related U.S. Application Data
(63) Continuation of application No. 10/619,520, filed on Jul. 14, 2003, now abandoned.
Application No. 10/619,520 is a continuation of application No. 09/462,446, filed on Jun. 5, 2000, now abandoned.

(30) Foreign Application Priority Data
Jul. 10, 1997 (DE) 197 29 591.6

(51) Int. Cl. A61K 39/44; C12M 1/24

(52) U.S. Cl. 424/178.1; 435/283.1

(57) ABSTRACT

The present invention relates to the use of a ligand for fibrinogen and/or fibrin for producing an agent for the treatment and/or prophylaxis of microcirculatory disorders and/or for influencing the rheology of a mammal.

The present invention relates to the use of a ligand for fibrinogen and/or fibrin for producing an agent for the treatment and/or prophylaxis of microcirculatory disorders and/for influencing the rheology of a mammal.

The present invention further relates to an adsorber column containing a matrix and a ligand, with the ligand having a specifity for fibrin and/or fibrinogen. Furthermore, the invention relates to a method for influencing the microcirculation of a mammal and to a pharmaceutical composition containing a ligand for fibrinogen and/or fibrin.

It has long been known that certain disorders and disease states are associated with the presence of an excess of a specific substance in a patient's blood. For instance, in hypercholesterolemia, the levels of low-density lipoprotein (LDL) in the patient's blood are greatly elevated due to a genetic defect in the LDL receptor. The elevation of LDL may lead to developing arteriosclerosis in the patient's coronary arteries, which in turn may lead to early cardiac infarction or death.

Certain autoimmune and other diseases also exhibit elevated levels of substances in the patient's blood. For instance, it is assumed that the symptoms of autoimmune diseases, such as systemic lupus erythematosus (SLE), rheumatoid arthritis, idiopathic thrombocytopenia, Myasthenia gravis and vasculitis, are caused by auto-antibodies and circulating immune complexes in the patient's blood which are directed against the patient's self-antigens. Thus, it has been assumed that the removal of a large portion of the patient's immunoglobulin, including auto-antibodies and circulating immune complexes (CIC), may lead to an amelioration of symptoms, and possibly a cure.

Interferon has also been discussed as a possible pathogenic substance in the blood of patients suffering from autoimmune diseases, allergy, and rejection of transplanted tissue. It has been proposed that anti-interferon immunoglobulins coupled to a solid support could effect the removal of interferon from the blood of such patients.

It is therefore possible to treat certain autoimmune diseases by removal of a significant portion of the patient's immunoglobulins using a column loaded with antibodies directed against human immunoglobulin. Use of such columns in the treatment of autoimmune diseases has e.g. been disclosed in the following documents: Schaumann D. et al., Nieren und Hochdruckkrankheiten, No. 9, September 1994; Knöbl P. et al., Thrombosis and Haemostasis, 74 (4), 1035-1038 (1995); Tribl B. et al., Ann. Hematology 71 (1995); Richter W.-O. et al., ASAIO Journal Vol. 451, No. 1, Suppl. P. 2 (1995); Schlee H. et al., Wiener Klinische Wochenschrift 108, Suppl. 1, 27 (1996); Dörffel et al., Zeitschrift für Kardiologie, Volume 85, Suppl. 2, Abstr. 667 (1996).

Another instance for removal of certain substances from a patient's blood arises in the case of a transplant. Generally, the transplanted organ must be immunologically matched to the recipient in order to prevent hyperacute rejection of the donor organ. However, if a donor organ is transplanted against which the recipient has formed or is forming antibodies, rejection of the donor organ follows rapidly after transplant. Such a reaction occurs when the recipient's own immune system attacks and destroys the transplanted organ within minutes to hours, typically within 48 hours after transplant. Even when the recipient receives immunosuppressive therapy, such a fast rejection cannot be prevented.

Methods have therefore been developed for removing anti-A/anti-B antibodies from the recipient's blood using extracorporeal perfusion of the recipient's plasma over synthetic A/B blood group antigens covalently linked to silica.

Furthermore, there are a number of diseases which have a deterioration of the microcirculation in common. Such a change may be the primary cause for bringing on the disease, or it may follow the disease. At any rate, it may essentially contribute to the clinical picture.

A reduced microcirculation may be caused by the vascular system in that e.g. inflammatory or metabolic changes reduce the vascular diameter of the arterioles, capillaries and venules thereby impairing microcirculation. The blood composition may also influence microcirculation. Of decisive importance are here the viscosity of the plasma and the deformability of the erthyrocytes. The blood composition also plays a role in disorders regarding macrocirculation; of decisive importance are here the viscosity of the whole blood and erythrocyte aggregation.

The plasma viscosity depends on the concentration of various macromolecules. For instance, fibrinogen, IgM, α₂-macroglobulin, and to a small degree chylomicrons, VLDL and LDL, influence the plasma viscosity in dependence upon the concentration.

The following survey shows the various complex components which influence the rheology of the blood and thus microcirculation:

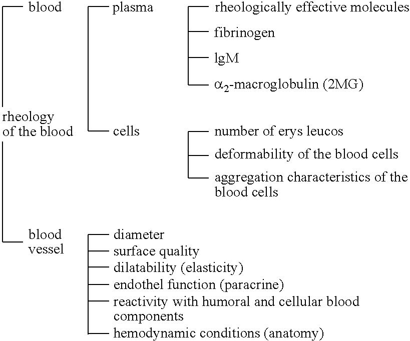

Apart from cardiac infarction, coronary cardiac death or apoplexy, there are a number of other diseases accompanying microcirculatory disorders. These include, for instance, type II diabetes. The group of diabetic persons is of particular importance, not least because of the high incidence and prevalence of diabetes. At the moment, about 4 million diabetics are living in the Federal Republic of Germany. About 15% to 25% have developed or are developing complications in the course of their disease, said complications being due to microcirculatory disorders.

These disorders include, e.g., diabetic foot, retinopathy, polyneuropathy and impaired kidney function. The importance, for instance, of the diabetic foot can already be gathered from the fact that in the United States of America there are hospitals exclusively specializing in the therapy of diabetic foot.

Further diseases accompanied by microcirculatory disorders are in particular arterial occlusive diseases, sudden deafness and sepsis. The following list shows diseases which may accompany microcirculatory disorders:

| | |
|---|---|
| CNS | apoplexy |
| | TIA (transient ischemic attack) |
| | PRIND (prolonged reversible ischemic neurological |
| | deficit) |
| | chronic vascular diseases of the CNS |
| | chronic intracranial circulatory disorders |
| | chronic extracranial circulatory disorders |
| | cerebrovascular circulatory disorders |
| | dementia |
| | Alzheimer's disease |
| | serious central vertigo |
| eye | chronic circulatory disorder |
| | acute vascular obliteration |
| ear | sudden deafness |
| | vertigo caused by the inner ear |
| | Morbus Menière |
| lung | primary pulmonary hypertonia |
| | veno-occlusive diseases of the lung |
| | thrombotic primary pulmonal hypertonia |
| | thromboembolic diseases of the large vessels |
| heart | transplantation vasculopathies |
| | acute myocardial infarction |
| | unstable angina pectoris |
| | small vessel disease of the heart |
| | non-operable serious coronary heart disease |
| | cardiomyopathies |
| abdomen | abdominal angina |
| kidneys | vasculopathies of the kidneys |
| | glomerulonephritis |
| | chronic kidney insuffiency |

peripheral arterial occlusive diseases

acute vascular occlusions

vasculitis

septic shock

disseminated intravascular coagulation (DIC) of other genesis, e.g. in the case of tumor diseases

type I+II diabetes
diabetic retinopathy
diabetic neuropathy
diabetic nephropathy

Up to now the possibilities of treating the above-mentioned diseases, in particular the accompanying microcirculatory disorders, have been limited.

For instance, diabetic gangrene has so far been treated in a purely symptomatic manner (bed rest, exact blood sugar adjustment, supportive therapy). Venous thromboembolisms are treated with heparin or by fibrinolysis therapy. In case of apoplexy aspirin may e.g. be used which acts by inhibiting the aggregation of blood platelets. Shock is e.g. treated with adrenergic agents, such as epinephrine. All of these agents, however, have in common that they are not suited for a really efficient prevention, nor do they exhibit any satisfactory results during treatment.

It has therefore been an object of the present invention to provide an efficient possibility of treating and influencing microcirculatory disorders and the rheology of blood. Another object of the present invention consists in providing an adsorber column which can be used for influencing the microcirculation of a mammal. Furthermore, it is an object according to the present invention to provide a method for influencing the microcirculation of a mammal. Finally, it is an object of the present invention to provide a pharmaceutical composition which is suitable for the treatment and/or prophylaxis of microcirculatory disorders.

These objects are achieved by the subject matters mentioned in the independent claims. Advantageous developments are indicated in the subclaims.

According to claim 1 of the present invention a ligand for fibrinogen and/or fibrin is used for producing an agent for the treatment and/or prophylaxis of microcirculatory disorders and/or for influencing the rheology of a mammal.

Ligand in this connection means a substance which specifically binds to fibrin and/or fibrinogen, the binding being preferably reversible.

Preferably, the ligand is a peptide which preferably comprises 3 to 10 amino acids. The peptide contains the following amino acid sequence in a particularly preferred manner:
Gly-Pro-Arg-Pro-X,
   wherein X may be any desired amino acid, such a lysin or polylysin or a spacer.

ε-aminocaproic acid or molecules with 6 C atoms are suitable spacers.

The following amino acid sequence has turned out to be particularly suited for the peptide:
Gly-Pro-Arg-Pro-Lys.

Further suitable sequences are:

Gly-Pro-Arg-X

Gly-Pro-Arg-Ser-NH₂

Gly-Pro-Arg-Val-NH₂

Arg-Gly-Asp-NH₂

Glu-His-lle-Pro-Ala-NH₂

Gly-Pro-Arg-Pro-Glu-Arg-His-Glu-Ser-HN₂

In a further embodiment of the present invention the ligand may be an antibody. It may be selected from polyclonal and monoclonal anti-fibrinogen antibodies and anti-fibrin antibodies.

In a further preferred embodiment the mammal is a human being.

Furthermore, in the agent the ligand may be bound to a solid matrix; the matrix may be selected from glass, carbohydrates, polymethacrylates and polyamides.

In a particularly preferred embodiment, the matrix is Sepharose.

The matrix may consist of beads, fibers and/or a membrane.

The diseases accompanying microcirculatory disorders may e.g. be diabetes, retinopathy, polyneuropathy, apoplexy, sudden deafness, sepsis, arterial occlusive diseases and/or an impaired kidney function.

Furthermore, the present invention is directed to an adsorber column which contains a matrix and a ligand, the ligand having a specifity for fibrin and/or fibrinogen. Preferably, the ligand is the peptide with the amino acid sequence
Gly-Pro-Arg-Pro-X,
   wherein X may be any desired amino acid or a spacer, and particularly preferred is a peptide with the amino acid sequence
Gly-Pro-Arg-Pro-Lys.

The matrix in the adsorber column is preferably Sepharose. The adsorber column may e.g. be prepared according to WO 95/31727.

Furthermore, the present invention is directed to a method for influencing the microcirculation of a mammal, wherein blood or plasma of the mammal is passed in vitro over the above-described column. An apheresis method is performed as the preferred method in which in a circuit blood is taken from the patient, said blood is separated into blood cells and plasma and passed over the adsorber column and subsequently returned to the patient.

Finally, the present invention is directed to a pharmaceutical composition which contains a ligand for fibrinogen and/or fibrin. It is possible thanks to the present invention to pass the blood of a patient with microcirculatory disorders over a column which contains e.g. Sepharose as the matrix and the above-mentioned peptide as the ligand, whereby fibrinogen and/or fibrin is removed from the blood. The blood can subsequently be returned to the patient, whereupon the fibrinogen and/or fibrin content in his blood is clearly reduced. It has been found that a reduced fibrinogen and/or fibrin content in the blood is directly accompanied by a reduction of microcirculatory disorders and thus by an improvement of the respective disease symptoms.

A hemorheologically effective decrease in the total amount of fibrinogen in the blood means a considerable improvement of the situation and considerably contributes to the treatment and/or prophylaxis of microcirculatory disorders. As a rule, this means a target value of 50 to 100 mg/dl of the patient's blood or a fibrinogen amount of 12 to 13.5 g to be removed per patient on the average. Therefore, an adsorber according to the double-column principle should have a binding capacity of about 2.5 to 3 g.

The above-mentioned values follow from high average fibrinogen concentrations of about 500 mg/dl and an assumed plasma volume of 3 l, whereby a total fibrinogen amount of about 15 g is calculated.

According to the present invention use is made of one or two adsorber columns by which the above-mentioned reduction can be achieved.

On account of the amounts of the fibrogen to be absorbed, which amounts are expected to be high, the double-column principle is preferably used in order
to limit the size of the adsorber,
to reduce the costs of the adsorber material,
whereby the plasma amount to be treated is unlimited.

On account of the circuit directly connected to the patient and of use of the double-adsorber principle with alternate loading and regeneration of the adsorber, that plasma amount can be desorbed that yields the desired fibrinogen reduction. As a rule, such a plasma loading amount should not be more than 1.5 to 2 times the plasma amount of the patient.

An adsorber size of less than 200 ml is preferably used.

The above-mentioned materials are suitable as adsorber material, Sepharose and membranes being particularly preferred.

The demand must be made on the ligands that it has a high affinity to fibrinogen and leads to a maximum unspecific reduction of 10 to 20% of coagulation factors, IgG, IgA, albumin, enzymes and hormones per session, a simultaneous absorption of IgM, macroglobulin, VLDL and LDL being also advantageous. This absorption, however, is always clearly below the percentage reduction of fibrogen because of the advantageous rheological effect to be expected therefrom.

A low hemodilution which is effected by substitution solutions also has an advantageous effect on the hemorheological parameters.

The peptide with the amino acid sequence
Gly-Pro-Arg-Pro-Lys
   has a very high specifity for fibrinogen and/or fibrin.

Further demands made on an adsorber column are that a material should be used that can easily be sterilized.

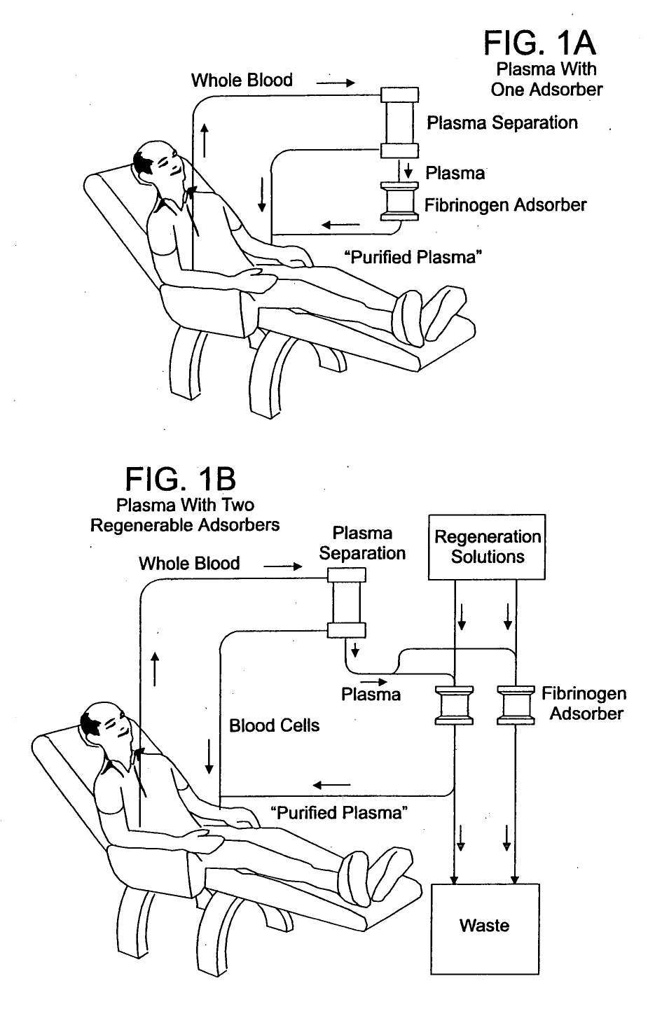

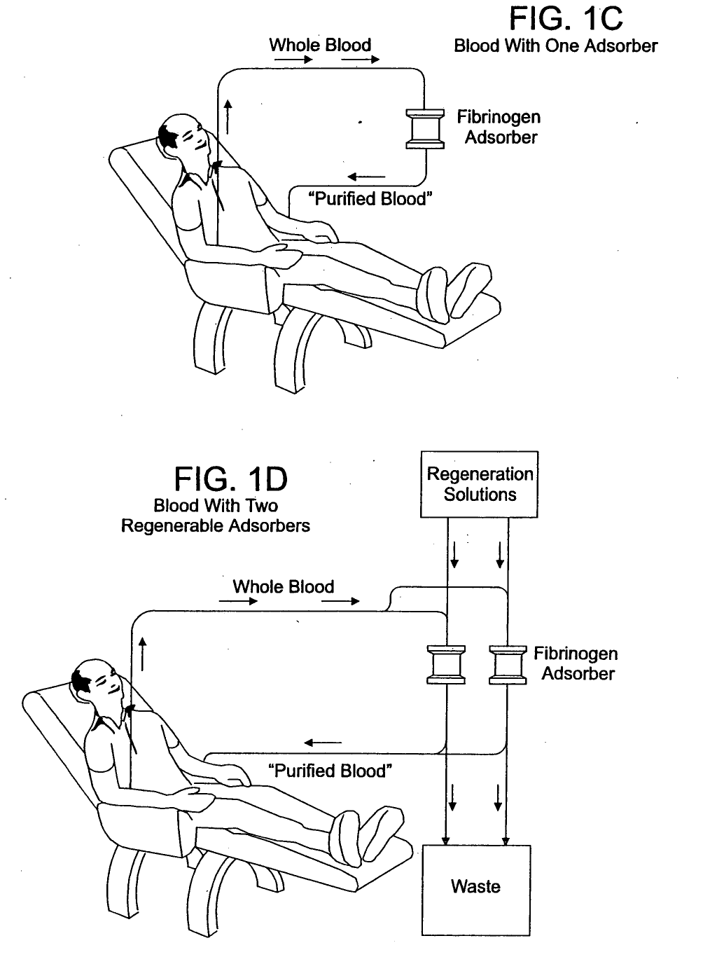

FIG. 1 (A-D) shows four examples of the use of adsorber columns according to the invention.

The subject matter of the present invention shall now be described in detail by way of the following examples:

### EXAMPLES

### Examples 1 and 2

The pentapeptide Gly-Pro-Arg-Pro-Lys was synthetized as trifluoracetate and coupled to cyanobromide-active Sepharose CL-4B. The specifity of the coupled Sepharose was tested by means of SDS gel electrophoresis. The material retained and subsequently eluted from the columns, and the standard fibrinogen preparation had identical bands.

Adsorption columns were prepared with the peptide-coupled Sepharose. 3 g Sepharose (wet weight) were used per column. The columns were first pre-rinsed with PBS and then with isotonic saline solution and then loaded with heparinized plasma (40 ml). After the column the plasma was collected in fractions of 3 ml each and the concentration of various plasma components and the plasma viscosity were measured in the samples. Subsequently, the columns were loaded with glycine-HCl buffer (pH 2.8) and the bound fibrinogen was thereby eluted. After loading with PBS and isotonic saline solution the columns could be loaded anew.

Measuring Methods:

Plasma viscosity was measured at 37° C. with a Contraves 30 low shear rotation viscosimeter.

Fibrinogen and immunoglobulins were immunonephelometrically measured with a Behring Laser Nephelometer. Cholesterol and triglycerides were enzymatically determined (Epos Autoanalyzer, Eppendorf, with reagents from Boehringer).

### Example 1

The following table shows the influence of the fibrinogen adsorber on the plasma concentration of fibrinogen and cholesterol and the resulting effect on the plasma viscosity (n=7).

TABLE 1

| | Fibrinogen | | |
|---|---|---|---|
| Fraction | (g/l) | Cholesterol (mmol/l) | Plasma Viscosity (mPas) |
| Plasma | 3.31 ± 0.20 | 6.40 ± 0.23 | 1.27 ± 0.02 |
| 1 | 0.94 ± 0.16 | 6.23 ± 0.17 | 1.17 ± 0.01 |
| 2 | 1.27 ± 0.17 | 6.29 ± 0.17 | 1.17 ± 0.01 |
| 3 | 1.49 ± 0.17 | 6.31 ± 0.16 | 1.18 ± 0.01 |
| 4 | 1.60 ± 0.15 | 6.32 ± 0.19 | 1.19 ± 0.01 |
| 5 | 1.81 ± 0.17 | 6.32 ± 0.18 | 1.20 ± 0.02 |
| 6 | 1.87 ± 0.16 | 6.29 ± 0.19 | 1.20 ± 0.02 |

The following table shows the influence of the fibrinogen adsorber on the plasma concentration of fibrinogen and triglycerides and the resulting effect on the plasma viscosity (n=7).

TABLE 2

| | Fibrinogen | | |
|---|---|---|---|
| Fraction | (g/l) | Cholesterol (mmol/l) | Plasma Viscosity (mPas) |
| Plasma | 4.29 ± 0.79 | 19.13 ± 7.04 | 1.42 ± 0.06 |
| 1 | 1.62 ± 0.70 | 16.28 ± 5.15 | 1.03 ± 0.05 |
| 2 | 1.90 ± 0.86 | 17.41 ± 5.86 | 1.22 ± 0.04 |
| 3 | 2.26 ± 0.92 | 17.54 ± 5.93 | 1.32 ± 0.05 |
| 4 | 2.52 ± 0.92 | 17.83 ± 5.97 | 1.34 ± 0.05 |
| 5 | 2.69 ± 0.90 | 18.12 ± 6.01 | 1.35 ± 0.05 |
| 6 | 2.85 ± 0.92 | 16.52 ± 5.15 | 1.33 ± 0.05 |

In both tests the fibrinogen reduction correlated with the plasma viscosity in a highly significant manner (paired sample T test).

### Example 2

Columns which were prepared in the same manner and which instead of the pentapeptide had a polyclonal anti-human-immunoglobulin sheep-derived antibody coupled to Sepharose CL-4B (specific binding of human IgG—all 4 subclasses—, IgM, IgA, immune complexes, fragments of immunoglobulins) also showed an effect on the plasma viscosity which, however, was definitely lower than with the fibrinogen adsorber.

The following table summarizes the results (n=7)

TABLE 3
Influence of an immunoglobulin adsorber on the plasma concentration of
fibrinogen, cholesterol, IgG, IgA, IgM
and the resulting effect on the plasma viscosity

| | | | | | | Plasma |
|---|---|---|---|---|---|---|
| | Fibrinogen | Cholesterol | IgG | IgA | IgM | Viscosity |
| Fraction | (g/l) | (g/l) | (g/l) | (g/l) | (g/l) | (mPas) |
| Plasma | 3.21 ± 0.20 | 6.40 ± 0.23 | 11.80 ± 0.43 | 2.96 ± 0.22 | 1.89 ± 0.25 | 1.27 ± 0.02 |
| 1 | 3.10 ± 0.20 | 6.25 ± 0.22 | 7.72 ± 0.73 | 2.77 ± 0.30 | 1.71 ± 0.27 | 1.21 ± 0.01 |
| 2 | 3.15 ± 0.21 | 6.30 ± 0.16 | 9.97 ± 0.57 | 2.87 ± 0.24 | 1.75 ± 0.28 | 1.24 ± 0.01 |
| 3 | 3.13 ± 0.21 | 6.28 ± 0.18 | 10.75 ± 0.51 | 2.88 ± 0.24 | 1.80 ± 0.29 | 1.25 ± 0.02 |
| 4 | 3.11 ± 0.20 | 6.25 ± 0.17 | 10.99 ± 0.45 | 2.83 ± 0.22 | 1.79 ± 0.29 | 1.24 ± 0.02 |
| 5 | 3.10 ± 0.20 | 6.30 ± 0.18 | 11.36 ± 0.45 | 2.85 ± 0.22 | 1.81 ± 0.29 | 1.25 ± 0.02 |
| 6 | 3.10 ± 0.19 | 6.17 ± 0.21 | 11.42 ± 0.44 | 2.86 ± 0.20 | 1.81 ± 0.30 | 1.25 ± 0.02 |

A significant change in the plasma viscosity is only seen in fractions 1 and 2 and corresponds to the lowest IgG values.

1. Use of a ligand for fibrinogen and/or fibrin for producing an agent for the in vitro treatment and/or prophylaxis of microcirculatory disorders and/or for influencing the rheology of a mammal.

2. Use according to claim 1, characterized in that the ligand is a peptide preferably having 3 to 10 amino acids.

3. Use according to claim 2, characterized in that the peptide contains the following amino acid sequence:
Gly-Pro-Arg-Pro-x

4. Use according to claim 3, characterized in that the peptide has the following amino acid sequence:
Gly-Pro-Arg-Pro-Lys

5. Use according to claim 1, characterized in that the liganc is an antibody.

6. Use according to claim 1, characterized in that the mammal is a human being.

7. Use according to claim 1, characterized in that the ligand is selected from polyclonal and monoclonal anti-fibrinogen antibodies and anti-fibrin antibodies.

8. Use according to claim 1, characterized in that the ligand in the agent is bound to a solid matrix.

9. Use according to claim 8, characterized in that the matrix is selected from glass, carbohydrates, polymethacrylates and polyamides.

10. Use according to claim 9, characterized in that the matrix is Sepharose.

11. Use according to claim 8, characterized in that the matrix consists of beads, fibers and/or a membrane.

12. Use according to claim 8, characterized in that the microcirculatory disorder appears in connection with diabetes, retinopathy, polyneuropathy, apoplexy, sudden deafness, sepsis, arterial occlusive diseases and/or impaired kidney function.

13. Adsorber column containing a matrix and a ligand, wherein said ligand has a specificity for fibrin and/or fibrinogen.

14. Adsorber column according to claim 13, wherein the ligand is a peptide containing the amino acid sequence: Gly-Pro-Arg-Pro-x wherein x can be any desired amino acid or spacer, or wherein the ligand is a peptide having the amino acid sequence Gly-Pro-Arg-Pro-Lys.

15. Adsorber column according to claim 13, wherein the matrix is Sepharose.

16. Method for influencing the microcirculation of a mammal, wherein blood of the mammal is passed in vitro over the column according to claim 13.

17. Method according to claim 16, characterized in that it is carried out as an apheresis method for plasma or whole blood.

18. Pharmaceutical compositions containing a ligand for fibrinogen and/or fibrin, wherein the ligand is a peptide having 3 to 10 amino acids.
